(19) United States
(12) Patent Application Publication
(10) Pub. No.: US 2023/0180802 A1
(43) Pub. Date: Jun. 15, 2023

(54) MICROWAVEABLE FROZEN LOBSTER TAIL CONTAINING CANNABIS AND METHOD OF DELIVERY OF CANNABIS COMPOUNDS

(71) Applicant: Alison W Barshak, Brookehaven, GA (US)

(72) Inventor: Alison W Barshak, Brookehaven, GA (US)

(21) Appl. No.: 17/463,485

(22) PCT Filed: Aug. 31, 2021
(86) PCT No.: PCT/US2021/048554

§ 371 Date: Aug. 31, 2021

(30) Foreign Application Priority Data
Aug. 31, 2020 (CA) 3091573

(51) Int. Cl. A23L 17/40; A61K 31/33; A61K 31/05; A23L 33/105; A23L 33/115; A23B 4/06; A23L 5/10; B65D 81/34; B65D 81/20

(52) U.S. Cl. CPC A23L 17/40; A61K 31/33; A61K 31/05; A23L 33/105; A23L 33/115; A23B 4/068; A23L 5/15; B65D 81/3461; B65D 81/2023; B65D 2581/3424

(57) ABSTRACT

In one embodiment, a minimally-processed microwaveable lobster product including a lobster tail, a fat-containing composition, and a microwaveable package. The lobster tail has a shell containing lobster meat. The shell has a longitudinal axis with at least two cuts formed generally parallel to the longitudinal axis, the tail meat is also split longitudinal. The fat-containing composition is disposed in the ventral region of the lobster tail. The microwaveable package has first and second resilient layers. The first resilient layer is disposed under the lobster tail and contacts the lobster tail. The second resilient layer is disposed above the lobster tail and contacts the lobster tail and the fat-containing composition.

In another embodiment the lobster tail is prepared with Cannabis missed with the fat-containing composition. This embodiment is similarly microwaveable.

### CROSS REFERENCE TO PRIOR APPLICATIONS

This application claims priority to CA 3,091,573 filed on Aug. 31, 2020; as well as, international application PCT/US21/48554 filed on Aug. 31, 2021; the contents of each application are hereby incorporated by reference in their entireties.

### FIELD OF THE DISCLOSURE

The present invention relates, generally, to shellfish, and more particularly, to the processing and cooking of lobster. The invention also relates to Cannabis and Cannabis compounds, including THC, THC-A and CBD oil. The invention further relates to the delivery of THC, THC-A and CBD oil.

### BACKGROUND

### Description of the Related Art

Consumers enjoy the flavor of fresh-cooked lobster. However, live lobster is an expensive, extremely perishable, and labor-intensive luxury item that has not been suited to home cooking and can be difficult to cook for the average home chef.

Some manufacturers fully cook lobsters (e. g., by steaming or sous-vide cooking) and then freeze or refrigerate and package the cooked lobsters, to provide a long shelf life and eliminate the requirement for consumers to cook the lobster at home. However, these fully-cooked lobsters are typically brined, often resulting in an overly salty or rubbery product. Or, due to sous-vide cooking methods, these lobsters are cooked at such a low temperature that the texture does not resemble that of a typical fresh-cooked lobster.

Alternatively, to improve the home cooking experience for consumers, some manufacturers sell whole lobster that has been blanched, i.e., partially cooked, prior to freezing and packaging. However, the blanching process cooks the juices out of the lobster and reduces its flavor, in like manner to boiling chicken.

Consumers can buy raw frozen lobster tails that the consumer thaws before cooking, but the thawing process causes loss of moisture and juices. Additionally, manufacturers use additives to keep these lobster tails plump, and these additives cause chemical reactions that tend to make sauces that these lobster tails are cooked in watery.

Other manufacturers sell lobster with meat that has been fully or partially loosened from the shell using high-pressure air or water, prior to freezing and packaging, so that the meat easily separates from the shell after cooking. However, the high pressure used to extract the lobster meat tends to degrade the texture and quality of the meat.

Consumers expect that cooking lobster in a microwave would result in tough, rubbery, and inedible lobster, which is the result normally obtained when attempting to cook raw lobster in a microwave. Until recently, there has previously been no way for a raw lobster by microwaving, in a single step, in a way that results in a tender, juicy, delicious, ready-to-eat product, with its own sauce, in a matter of minutes.

However, in contrast to the above here-to-for assumption, U.S. Pat. No. 10,806,154 to Alison W Barshak (also the inventor of the present invention), demonstrates that a technique exists to prepare frozen lobster tail such that microwaving them creates an edible—and delicious—product while retaining a substantial portion of the natural fluid of the lobster tail. The disclosure of U.S. Pat. No. 10,806,154 issued on Oct. 20, 2020 is hereby incorporated by reference in its entirety.

Meanwhile, there are many people who suffer from both chronic pain and suppressed appetite. For example, cancer patients, Crohn's disease patients and people with chronic depression all commonly exhibit symptoms of chronic pain and low appetite. While Cannabis, particularly the active pharmaceutical, TCH, may be able to act as an analgesic to relieve the pain of these patients, the most common dosing means—inhalation of combusted Cannabis plants—is unattractive. Likewise, other edible forms of delivery of TCH may likewise be unattractive.

There exist a need for a method of supplying an effective dose of THC through a simple and attractive edible that overcomes the low appetite impediment of existing THC containing edibles.

Here is presented a combination of a microwavable lobster tail that is prepared with Cannabis—or Cannabis products—within, or in proximately of, the fat-containing substance that is an element of the microwaveable lobster tail.

### SUMMARY OF THE INVENTION

Embodiments of the invention provide solutions to the foregoing problems and additional benefits, by providing a way for an ordinary consumer with a microwave to enjoy lobster tail at home and a processing and cooking method for fresh-frozen lobster that is quick, neat, easy, labor-reduced, waste-reduced, self-contained, and results in a perfectly-cooked, ready-to-eat lobster product that tastes the same as if cooked from live in a conventional manner.

In one embodiment, the present invention provides a minimally-processed microwaveable lobster product including a lobster tail, a fat-containing composition, and a microwaveable package. The lobster tail has a shell containing lobster meat. The shell has a longitudinal axis with at least one cut formed generally parallel to the longitudinal axis. The fat-containing composition is disposed in a region above the lobster tail. The microwaveable package has first and second resilient layers. The first resilient layer is disposed under the lobster tail and contacts the lobster tail. The second resilient layer is disposed above the lobster tail and contacts the lobster tail and the fat-containing composition.

In another embodiment, the present invention provides a method for manufacturing a microwaveable lobster product. The method includes: (a) forming a cut generally parallel to the longitudinal axis of the shell of a lobster tail containing lobster meat; (b) disposing a fat-containing composition in a region above the lobster tail; and (c) disposing the lobster tail and fat-containing composition together in a microwaveable package comprising first and second resilient layers, such that the first resilient layer is disposed under the lobster tail and contacts the lobster tail, and the second resilient layer is disposed above the lobster tail and contacts the lobster tail and the fat-containing composition.

### BRIEF DESCRIPTION OF FIGURES

FIG. 1 shows a flowchart of an exemplary method for manufacturing a microwaveable frozen lobster tail product consistent with a first exemplary embodiment of the invention;

FIG. 2 shows a lobster tail section, in a step of the exemplary method of manufacture of a microwaveable frozen lobster tail product consistent with a first exemplary embodiment of the invention;

FIG. 3 shows a lobster tail section with a cut made lengthwise in the shell, in a step of the exemplary method of manufacture of a microwaveable frozen lobster tail product consistent with a first exemplary embodiment of the invention;

FIG. 4 shows a lobster tail section with the lobster meat pulled up through the cut in the shell to expose a portion of the meat, in a step of the exemplary method of manufacture of a microwaveable frozen lobster tail product consistent with a first exemplary embodiment of the invention;

FIG. 5 shows a lobster tail section with a fat-containing composition placed above the lobster tail, in a step of the exemplary method of manufacture of a microwaveable frozen lobster tail product consistent with a first exemplary embodiment of the invention;

FIG. 6 shows a two-piece microwaveable thermoform pouch adapted to vent during cooking, in a step of the exemplary method of manufacture of a microwaveable frozen lobster tail product consistent with a first exemplary embodiment of the invention;

FIG. 7 shows a finished microwaveable frozen lobster tail product consistent with a first exemplary embodiment of the invention;

FIG. 8 shows a finished microwaveable frozen lobster tail product consistent with a second exemplary embodiment of the invention;

FIG. 9 shows a finished microwaveable frozen lobster tail product consistent with a third exemplary embodiment of the invention;

FIG. 10 shows an exemplary application of a microwaveable frozen lobster tail product consistent with a fourth exemplary embodiment of the invention;

FIG. 11 shows an assembly line with lobster tails cut twice lengthwise through both the dorsal and ventral sides of the shell, as well as slice through the lobster tail meat;

FIG. 12 shows the prepared lobster tail—with lobster tails cut twice lengthwise through both the dorsal and ventral sides of the shell, as well as slice through the lobster tail meat—after microwaving, the liquefied butter now a sauce, clearly visible.

FIG. 13 shows the chemical structure of THC;

FIG. 14 shows the chemical structure of CBD; and,

FIG. 15 shows the decarboxylation reaction converting THCA to THC.

### DETAILED DESCRIPTION OF THE INVENTION

### Definitions

The term “lengthwise,” as used herein, refers to a direction generally parallel to the longitudinal axis of the lobster tail.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments.

The term “ounce”, abbreviated “oz.” is used throughout the disclosure and is intended to mean the English unit of measure for mass, equal to about 28.3495 grams.

As used here, the terms “THCA” and “Tetrahydrocannabinolic acid” are used interchangeably. Both terms as used herein are meant to refer to the chemical tetrahydrocannabinolic acid. Tetrahydrocannabinolic acid is a precursor of tetrahydrocannabinol (THC), an active component of Cannabis. In other literature THCA is also referred to as THCA, 2-COOH-THC; conjugate base tetrahydrocannabinolate these terms are considered synonymous with TCHA for the purpose of this disclosure.

The terms “THC” and “Tetrahydrocannabinol” are used interchangeably herein. Both terms refer to the chemical compound tetrahydrocannabinol and its cannabinoid isomers. Tetrahydrocannabinol along with its double bond isomers and their stereoisomers are commonly found in Cannabis. THC (including THC isomers) is the principal psychoactive constituent of Cannabis. Particularly, dronabinol, a pharmaceutical form of THC is included as a chemical compound referred to herein as THC.

As used herein the terms “CBD”, “CBD oil” and “Cannabidiol” (CBD) are terms used interchangeably herein. Each terms refers to the chemical compound “cannabidiol” is a phytocannabinoid commonly found in Cannabis plants.

The term “Cannabis” as used herein collectively refers to any one or combination of the plant Cannabis sativa, Cannabis indica, Cannabis ruderalis. Specific reference to a species of Cannabis refers specifically to that species. Specific reference to a strain and/or cultivar of a Cannabis species specifically refers to the named strain and/or cultivar. Cannabis is also known as “marijuana”. There are other terms for Cannabis that are used in the art and society that are known to refer to the Cannabis plant, its flower and or the active compounds of Cannabis. Those terms are too numerous and fluid to recite here but are recognized by those of skill in the art.

Packaging and Cooking of Lobster Tails

Microwaving thawed lobster normally results in uneven cooking and tough, rubbery lobster meat. Instead, embodiments of the invention use fresh-frozen lobster designed to be cooked in the microwave while still frozen, to avoid this problem. The lobster is cooked from frozen inside microwaveable packaging, along with a fat-containing composition. In some embodiments, various herbs, spices, seasonings, and flavorings are included in the fat-containing composition to form a sauce that combines the juices of the lobster with these other elements. The sauce is created while the lobster tail cooks inside the packaging. The flavors can be customized, and since the lobster is cooked together with the fat, the combination of lobster juice and sauce forms an integrated lobster-and-sauce product, with no loss of juices or flavor. The natural juices from the raw lobster are captured and infused into the sauce, and the sauce is created as the lobster cooks, rather than after the lobster cooks.

Embodiments of the invention provide a new type of minimally-processed product for the lobster industry for an entire new market, creating new demand, and increasing revenue for lobstermen and seafood companies, while decreasing transportation and storage costs. Embodiments of the invention eliminate the problem of attempting to provide live lobsters that often die during transport or at some other point before cooking. Additionally, live lobsters held in a tank lose weight over time, and embodiments of the invention avoid this problem by fresh-freezing lobster tails instead of storing live lobsters in a tank.

Conventional processing of lobster for preparation by the home consumer involves the use of considerable amounts of sodium. Although a certain amount of sodium is naturally present in shellfish, the presence of an excess amount of sodium in shellfish normally does not interfere with home cooking, such as when shellfish is boiled, baked, fried, grilled, or the like.

However, when shellfish is cooked in a microwave oven, an abundance of sodium can be problematic. Microwave ovens cook food primarily by reversing the polarity of water molecules in food each millisecond, thereby creating friction that cooks the food as the water molecules essentially act as fuel sources. Although protein, fat, and starch molecules also absorb microwave energy to a lesser degree, salt molecules tend to break apart in the presence of water. When this occurs, sodium and chlorine ions create heat by colliding in the rapidly-oscillating electromagnetic field, leaving less microwave energy available to cook the center of the food. A result of this phenomenon can be seen with a refrigerated bowl of soup placed in a microwave for reheating. The soup near the outside of the bowl encounters the microwave energy first, heats rapidly, and appears to boil. However, the center of the soup is still cold, because the salt ions around the outside of the bowl absorb sufficient energy to produce localized boiling before the rest of the soup can even absorb sufficient energy to begin heating.

When shellfish containing too much sodium is cooked in a microwave, the meat becomes mushy due to the breakdown of the protein structure caused by the abundance of sodium ions. For this reason, shellfish treated with sodium tripolyphosphates (STPPs), shellfish that has been brined, and shellfish that contains too much sodium for any other reason is not suitable for microwave cooking. Accordingly, conventional lobster products, which are mostly brined and treated with STPPs, are unsuitable for use in connection with microwave cooking.

It is therefore desirable that no brining steps be included during the flash-freezing process, nor in any other part of the process of manufacturing a microwaveable frozen lobster tail product consistent with embodiments of the invention. This is because brine is a salt-based solution, and an abundance of salt ions interferes with microwave cooking.

For similar reasons, lobster treated with STPPs or other sodium salts should also desirably not be used in any part of the process of manufacturing a microwaveable frozen lobster tail product consistent with embodiments of the invention.

For similar reasons, any fat-containing composition used in a lobster product consistent with embodiments of the invention should desirably contain little or no salt. For example, if butter is used as a fat-containing composition, then the butter should desirably be unsalted butter, or composed primarily of unsalted butter, i.e., unsalted butter with only a small amount of salt added for taste, e.g., a concentration of 95% unsalted butter or higher.

While normal processing of lobster includes brine and STPPs, minimal processing of lobster according to embodiments of the invention does not include steps of brining or the use of STPPs.

Avoiding the use of brine, STPPs, and/or fat-containing compositions that contain salt ions, therefore, promotes evenness of cooking when the lobster tail is cooked in the microwave.

Embodiments of the invention provide processes for manufacturing and cooking a lobster tail, as well as lobster-tail products manufactured and cooked according to such processes.

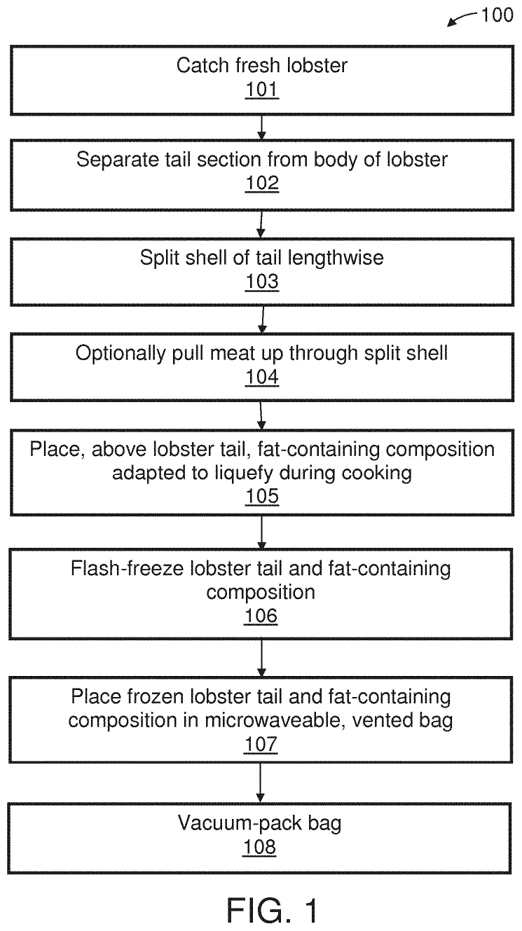

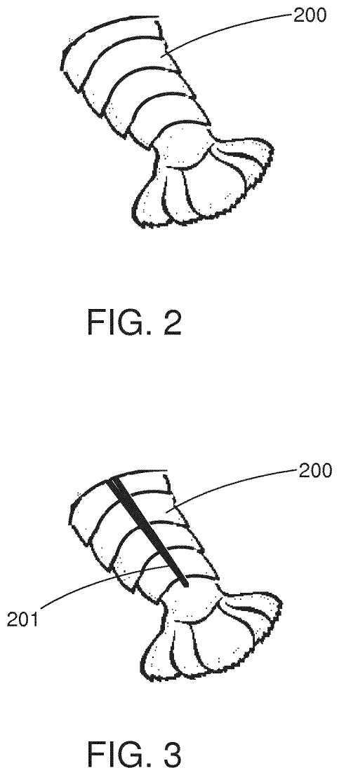

With reference to the flowchart of FIG. 1 and the views of FIGS. 2-7, an exemplary method 100 for manufacturing a minimally-processed microwaveable frozen lobster tail product consistent with a first exemplary embodiment of the invention will now be described.

First, at step 101, a fresh lobster is caught. As shown in FIG. 2, at step 102, a lobster tail section 200 with the shell intact, weighing approximately 3.75 to 4 ounces (106 to 113 grams), is separated from the body of the lobster.

As shown in FIG. 3, at step 103, a cut 201 is made in at least a portion of the shell of the lobster tail, to split the shell longitudinally. Although FIG. 3 shows that a split is made only along the top surface of the lobster tail, it is recommended that two lengthwise are made along the top AND bottom surfaces of the tail to split the shell to the fan. Likewise, it is recommended the tail meat is split longitudinally. These cuts can be made in two or three separate cuts or in a single cut. As long as both the dorsal and ventral sides of the shell, as well as the tail meat are cut generally longitudinally the majority of the length of the tail the shell and tail meat will be prepared for the practice of the invention as described herein.

Splitting or cutting the shell and tail meal as described makes the lobster tail meat easier to remove from the shell and therefore easier to eat. However, importantly also results in the lobster meat cooking in a sauce that will be created during the cooking process, as will be explained in further detail below, which imparts additional moisture into the meat.

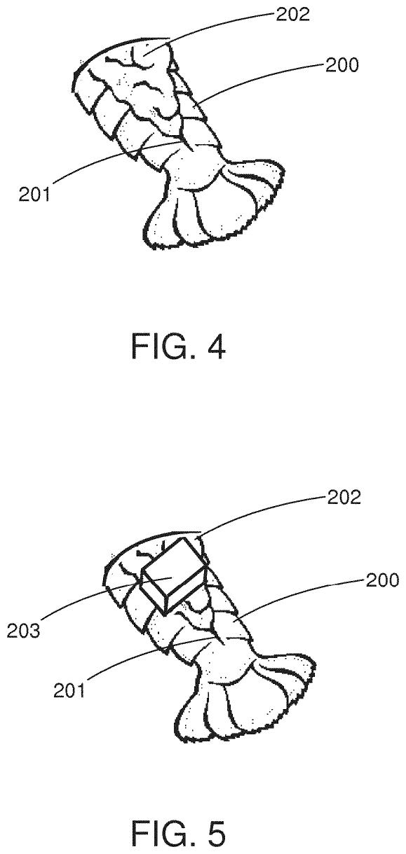

As shown in FIG. 4, at an optional step 104, after the shell has been split, the lobster meat 202 is pulled up through the cut 201 in the shell of the lobster tail, so that a portion of the meat 202 is exposed outside of the shell.

As shown in FIG. 5, at step 105, a solid or semi-solid composition 203 containing a fat-adapted to liquefy during cooking, such as 1 ounce (28 grams) of butter or compound composed of mostly butter, is placed above the lobster tail meat on the ventral side of the tail. Placement on the ventral side of the lobster is critical to the proper distribution of the fat-containing composition. In one embodiment, the fat-containing composition is disposed above or near an exposed portion of lobster meat, so that the liquid will trickle directly onto the meat as it liquefies during cooking. The fat-containing composition may further contain herbs, spices, seasonings, and other flavorings that will be infused into the lobster meat during cooking. Cannabis compounds may be added to the fat-containing composition.

At step 106, the lobster tail and fat-containing composition are flash-frozen. Flash freezing (or “blast freezing”) freezes the lobster tail quickly by subjecting it to cryogenic temperatures, e.g., well below negative 18° C. (0° F.) well below the melting point of water, causing the water inside the lobster tail to freeze in a very short period without forming large crystals, thus avoiding damage to sensitive cell membranes. It is advised that the lobster must be maintained below about negative 18° C. (0° F.) at this point and forward throughout further packaging, labeling, shipping and point of sale until transfer to the customer end-user. More preferably, the lobster products are maintained at a temperature of between about negative 20° C. to about negative 80° C. (−4° F. to −112° F.) after initial freezing. More preferably, the lobster products are maintained at a temperature of between about negative 25° C. to about negative 75° C. (−13° F. to −103° F.) after initial freezing. Most preferably, the lobster products are maintained at a temperature of between about negative 26° C. to about negative 30° C. (−15° F. to −20° F.) after initial freezing.

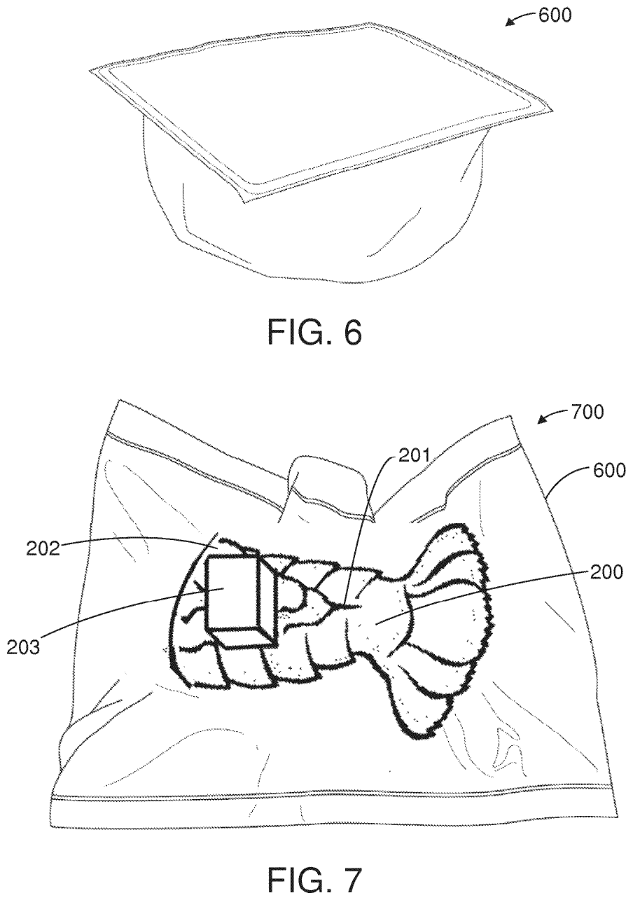

At step 107, while maintaining the temperature at least below about negative 18° C. (0° F.), the frozen lobster tail and fat-containing composition are placed in a two-piece microwaveable thermoform pouch, such as the exemplary pouch 600 shown in FIG. 6. Pouch 600 is formed from two layers of resilient material that is strong enough to prevent being pierced by the lobster shell, and it is specifically noted that the lobster tail does not rest on any kind of rigid carrier or pallet within pouch 600. Additionally, the top resilient layer of pouch 600 directly contacts the fat-containing composition, which remains above the lobster tail as the lobster tail is cooked. In this manner, both the top and bottom resilient layers of pouch 600 make direct contact with the lobster tail, and there is no risk of interference caused by any additional rigid carrier or pallet layer during the cooking process. An additional cost saving is achieved by not having to use an additional rigid carrier layer or tray to hold the lobster tail within pouch 600.

At step 108, the contents of the pouch are vacuum-packed, resulting in product 700 shown in FIG. 7, which is ready for (optional) additional packaging, and then labeling and transport to a retail supply chain.

The home consumer purchases a unit at retail that contains product 700 and brings it home. To cook the lobster tail, the consumer pierces product 700 with the tip of a knife (e.g., at the location of the fat-containing composition) for ventilation, then inserts product 700 into a standard microwave oven, shell-side down, and cooks it at a power of 800 watts for approximately 2 minutes and 30 seconds, to an internal temperature of at least 60° C. (140° F.), more preferable between 60° C. (140° F.) and 65° C. (150° F.), most preferably 62° C. (145° F.). It was previously reported to cook the lobster tails to 84.4° C. (184° F.); however, it has been subsequently found that this high temperature damages the lobster tail meat leaving a rubbery taste. It is also not necessary to use such high temperatures to ensure a safe and sanitary product. The above ideal temperature range should be used when preparing the disclosed invention

End users should keep in mind, at higher wattages, small downward adjustments in cooking times ought to be made. During the cooking process, the frozen lobster tail defrosts and gently poaches in the microwaveable pouch to fully cook, while the fat-containing composition liquefies and forms a sauce that combines with the lobster meat to create a tender and juicy ready-to-eat lobster tail. While many methods of cooking raw lobster tail, such as grilling, result in the cooked lobster tail sticking to the shell, an advantageous and convenient result of microwave-cooking a lobster tail is that the lobster meat separates easily from the shell, in a single piece.

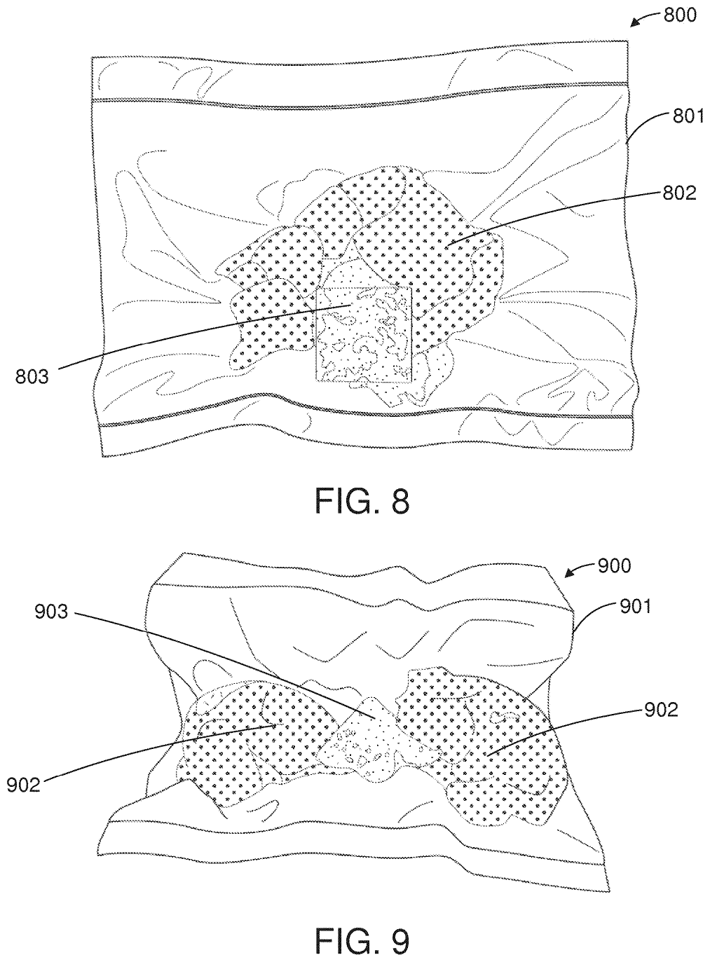

FIG. 8 shows a microwaveable frozen lobster tail product 800 consistent with a second exemplary embodiment of the invention. As shown, product 800 is similar to product 700, except that product 800 employs a lobster tail 802 weighing approximately 2.25 to 3 ounces (64 to 85 grams) with its shell fully removed. A fat-containing composition 803 is disposed above the shelled lobster tail 802, either before or after flash-freezing, and the lobster tail 802 and fat-containing composition 803 are vacuum-packed into a two-piece microwaveable pouch 801 adapted to vent during cooking. Although FIG. 8 shows a whole lobster tail in a package, a shelled lobster tail may be cut into two or more pieces prior to packaging in some embodiments. To cook the lobster tail, the home consumer inserts product 800 into a standard microwave oven, with the underside of the tail desirably facing up, and cooks it at a power of 800 watts for approximately 1 minute and 30 seconds, at an internal temperature of at least 60° C. (140° F.), more preferable between 60° C. (140° F.) and 65° C. (150° F.), most preferably 62° C. (145° F.) At higher wattages, small downward adjustments in cooking times are made.

FIG. 9 shows a microwaveable frozen lobster tail product 900 consistent with a third exemplary embodiment of the invention. As shown, product 900 is similar to product 800, except that product 900 employs two lobster tails 902 with their shells fully removed, packed together in a single two-piece microwaveable pouch 901. A single portion of fat-containing composition 903 is disposed centrally above and located between the two lobster tails 902, either before or after flash-freezing, and the lobster tails 902 and fat-containing composition 903 are vacuum-packed into the pouch 901. This arrangement permits the use of a single portion of fat-containing composition with multiple lobster tails. Although FIG. 9 shows lobster tails 902 with their shells fully removed, this arrangement can also be used with lobster tails that are still in their shells. Although FIG. 9 shows only two lobster tails packaged together, it should be understood that more than two lobster tails could be packaged together, in alternative embodiments of the invention. However, it has been found that a total weight of lobster not exceeding 8 ounces (or 226 grams) in a single microwaveable package is desirable in order to consistently produce a high-quality product when cooked. Accordingly, in one embodiment, four 2-ounce (57 gram) lobster tails are desirably disposed in a single microwaveable package. In another embodiment, two 3-ounce (85 gram) lobster tails are desirably disposed in a single microwaveable package.

It is desirable that a ratio of approximately 1 ounce (28 grams) of fat-containing composition be used either per 3-ounce (85-gram) lobster tail, or for every 3 ounces (85 grams) of lobster meat, and that the amount of fat-containing composition can be increased or decreased appropriately to suit the amount of lobster meat used. The fat-containing composition is desirably placed on the flesh side of the lobster tail.

In some embodiments, the fat-containing composition is added after the lobster tail is flash-frozen instead of the fat-containing composition being flash-frozen along with the lobster tail.

In some embodiments, a fat-containing liquid may be used, instead of a fat-containing solid or semi-solid adapted to liquefy during cooking. In this scenario, the lobster may absorb the liquid so as to marinate before being flash-frozen, whereby the infusion of fat into the lobster is similar to the process that takes place while marinating a piece of raw chicken or other meat.

In some embodiments, the packaging is vented and includes one or more seams adapted to vent at a predetermined temperature and/or pressure. Alternatively, the packaging may be fully sealed, and the consumer may be directed to pierce, slit, or otherwise vent the packaging prior to microwave cooking. In other embodiments, non-vented packaging is used, in which case bursting a seam is not likely to affect doneness. However, the user is directed to avoid a mess in this instance by placing the package on a plate or bowl during cooking.

It is desirable that no water glaze be included during the flash-freezing process, nor in any other part of the process of manufacturing a microwaveable frozen lobster tail product consistent with embodiments of the invention. This is because water glaze tends to produce a waterlogged sauce and a lobster with a significant reduction in lobster flavor. Further, water glazing increases the price of lobster meat to the consumer, due to the addition of unnecessary water weight.

Variables that may affect microwave cooking time for the packaged lobster tail meat include, e.g., microwave oven wattage, preparation of the lobster tail (shell on/shelled; whole/cut into pieces; etc.), lobster tail size and weight, contents of the fat-containing composition, type of microwavable packaging and ventilation scheme employed, and the like.

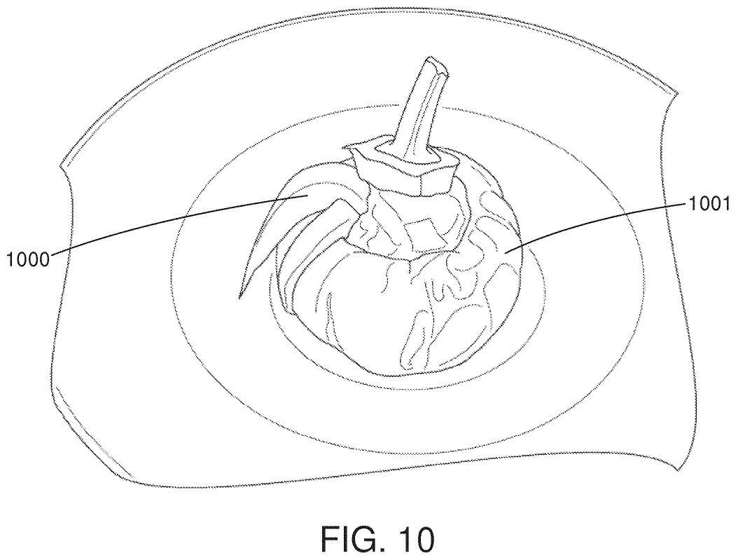

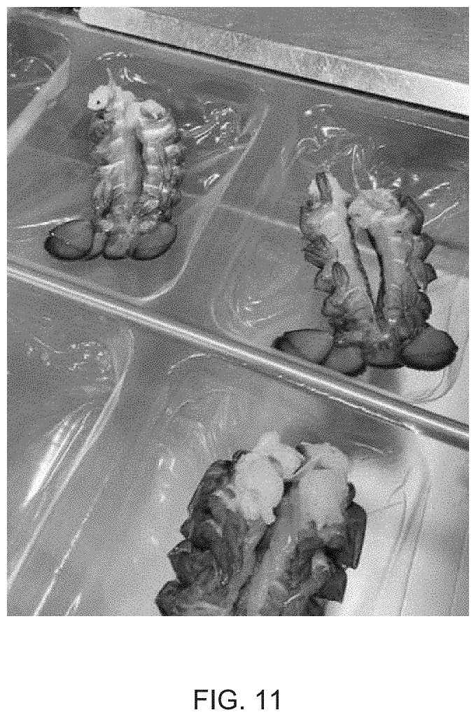

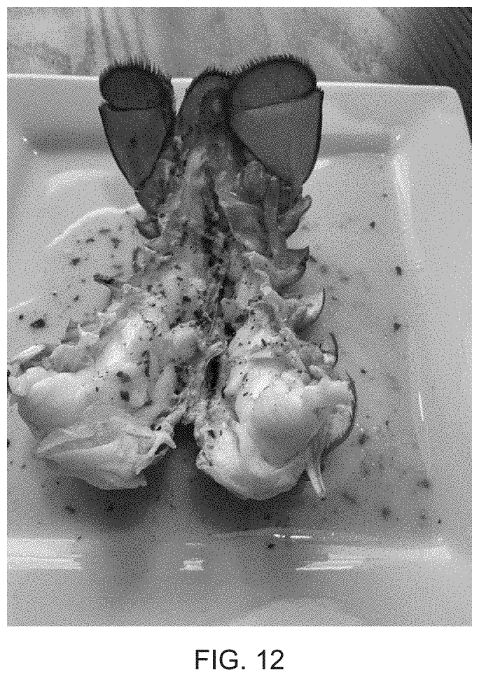

The lobster tail product can be adapted by a manufacturer for specific applications. As shown in FIG. 10, in a fourth exemplary embodiment, the lobster tail product is pre-combined with specific flavorings in the thermoform packaging to make a one-step microwaveable filling 1000 for a gourd 1001.

The tails of either soft-shell lobsters or hard-shell lobsters may be used, in different embodiments of the invention. Soft-shell lobster tails are relatively easier to use in conjunction with thermoform packaging because the incidence and risk of piercing the packaging is reduced relative to hard-shell lobster tails, which have hard edges and may require repackaging after piercing. Additionally, the use of smaller lobster tails (e.g., up to 8 ounces (or 226 grams) for soft shells in summer, and up to 4.5 oz (or 113 grams) for hard shells in winter) is desirable because the shells are softer relative to larger lobster tails. Additionally, soft-shell lobsters are juicier due to a higher water content and tend to produce a more tender final product. It is also desirable that tails of the North American Lobster (Homarus americanus) be used in embodiments of the invention.

A product consistent with embodiments of the invention has numerous advantages, including the following. There is a saving in labor, both in terms of eliminating the need to keep lobster alive and properly conditioned, and reducing kitchen preparation time and effort. The cooked lobster tail product has a restaurant-quality consistency and texture and a quality flavor. There is no need to dip the cooked lobster in sauce because the lobster emerges from the cooking process already combined with the sauce. Lobster out of the shell and split tails in the shell can expand during cooking without the restrictions that would occur if the lobster were completely enclosed by the shell. The lobster meat plumps and is better able to absorb the accompanying fat and additional flavoring when cooking from frozen.

Embodiments of the invention are suitable for the home cook who wants to have perfectly-cooked lobster as part of a meal in a short amount of time, without the mess, investment in additional ingredients and tools, time and money associated with conventional lobster cooking, and further providing the benefit of marrying a sauce with the lobster, using the lobster's original juices. The product can be used by end consumers with little knowledge of how to properly cook lobster.

In alternative embodiments, previously-frozen lobster tails could be used instead of fresh-frozen or flash-frozen lobster tails, depending on the methods used to process those tails and the degree of quality intended for the end product. Price may be an issue for the end consumer, and a previously-frozen tail may end up being less expensive to use than a tail processed from fresh.

It is also noted that lobsters lose weight when out of water. Over a 24-hour period, a 3-4% loss of weight can be observed, which results in less sauce after cooking using methods such as those exemplary methods described herein.

It should further be noted that adding a fat-containing composition to a raw lobster tail before freezing is likely to produce a more flavor-infused tail meat, while adding such butter to an already-frozen tail is likely to reduce the infusion of flavor into the lobster meat relative to a fresh tail, whose juices will combine with the fat-containing composition to create an extremely flavorful sauce.

It has been observed by the inventor hereof that, for lobster tails with shell on, using an 1100-watt continuous-cook microwave oven, the following cooking times are desirably used, in embodiments of the invention:

| Lobster tail weight | Cook Time |
|---|---|
| 3 oz. (85 grams) | 2 minutes 45 seconds |
| 4 oz. (113 grams) | 3 minutes |
| 4 to 4.5 oz. (113 grams to 126 grams) | 3 minutes 15 seconds |
| 5 to 5.35 oz. (142 to 152 grams) | 3 minutes 25 seconds |

It has also been observed by the inventor hereof that, in one embodiment of the invention, a shell-on lobster tail weighing 3.86 ounces (109 grams) with 1.108 ounces (31.4 grams) of butter cooked well in an 1100-watt continuous-cook microwave oven in 2 minutes 45 seconds in a vented package, with a good texture and flavor. In another embodiment of the invention, a shell-on lobster tail weighing 6.07 ounces (172 grams) with 1.108 ounces (38.8 grams) of butter cooked well in an 1100-watt continuous-cook microwave oven in 4 minutes in a vented package, with a good texture and flavor. In another embodiment of the invention, a shell-on lobster tail weighing 6.19 ounces (175 grams) with 1.108 ounces (41.3 grams) of butter cooked well in an 1100-watt continuous-cook microwave oven in 4 minutes in a vented package, with a good texture and flavor.

Cannabis

It is recognized that the microwavable lobster tails of the disclosed invention is a particularly good vehicle for the oral delivery of Cannabis and Cannabis compounds including THC and CBD oil. It is also recognized that the microwave cooking of microwavable lobster tails of the disclosed invention is a particularly effective process for the activation of THC and CBD oil within—or extracted from—Cannabis.

The microwave cooking of the disclosed microwaveable lobster tails is found to be effective at both decarboxylation of THCA into pharmaceutically active THC and preserving pharmaceutically active THC during the heating step of food containing pharmaceutically active THC.

The microwave cooking of the disclosed microwaveable lobster tails is found to be effective at both decarboxylation of CBD oil into active CBD oil and preserving active CBD during the heating step of food containing active CBD oil.

Decarboxylations

Decarboxylation is the process that activates compounds in Cannabis such as THC and CBD oil. All cannabinoids contained within the trichomes of raw Cannabis flowers have an extra carboxyl ring or group (COOH) attached to their chain. For example, tetrahydrocannabinolic acid (THCA) is synthesized in prevalence within the trichome heads of freshly harvested Cannabis flowers. THCA, in most strains, prevails as the highest cannabinoid present in items that have not been decarboxylated.

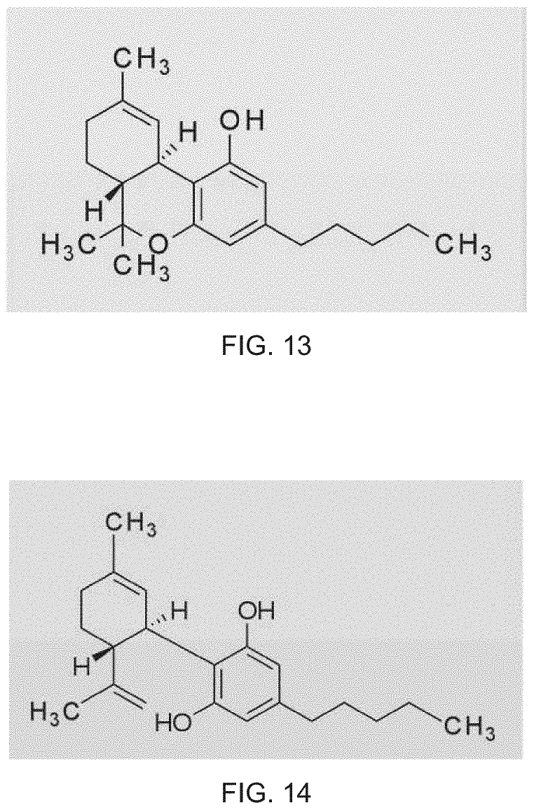

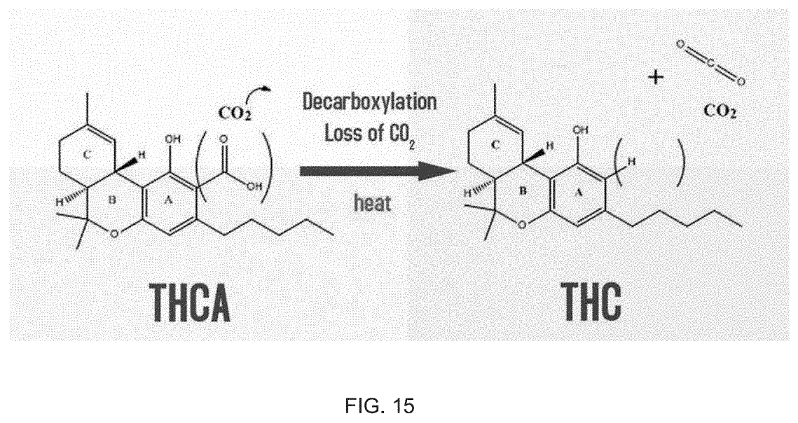

THCA has a number of known benefits when consumed, including having anti-inflammatory and neuroprotective qualities. But THCA is not psychoactive. For Cannabis leaves and/or buds to have a psychoactive effect, THCA must be converted into THC through decarboxylation, see FIG. 13.

The two main catalysts for decarboxylation to occur are heat and time. Simple drying of Cannabis at room temperature over days at room temperature will convert some THCA to THC and CBDA to CBD, generally, the conversation at room temperature is incomplete despite the long length of time. One the other extreme, high temperatures cause decarboxylation to occur in seconds with a yield approaching 100%. However, pyrolysis destroys about a third of available THC reducing the yield to 60-70% of available THCA. Both smoking and vaporizing will instantaneously decarboxylate cannabinoids due to the extremely high temperatures present (typically about 300° C. to about 700° C.). However, the high temperatures also volatilize the cannabinoids. Therefore, when decarboxylated at extremely high temperatures the THC and other cannabinoids, must be inhaled to be consumed.

To consume Cannabis leaves and buds orally and achieve a psychoactive effect from a clinical dose of THC decarboxylation has to occur below the temperature of vaporization. Heating cannabinoids at a lower temperature over greater time allows decarboxylation of the cannabinoids while avoiding vaporization of the material. Most midrange temperatures about 80° C. (176° F.) to about 145° C. (293° F.)—require 7 to 60 minutes for high yield decarboxylation of THCA to THC. Metabolic processes during oral consumption are anticipated to reduce the yield of THC by 4-12%, versus a ˜33% loss due to combustion at high temperatures.

THC and CBD are well known to be highly lipophilic. Both dissolve readily in fatty substances. Lipids are also known to stabilize decarboxylated cannabinoids.

Cannabis Added to Microwaveable Lobster Tails

It is observed that raw Cannabis can be added to the fat-containing composition of the microwavable lobster tails product described here to deliver a clinical psychoactive dose of THC when the product is consumed after microwaving.

Harvested untreated Cannabis bud and leaf may be added to the butter or other fat-containing composition used in the preparation of the described microwavable lobster tail. It is recommended that a single dose of Cannabis material is added to a single lobster tail product. The Cannabis material is best added to the butter or other fat-containing composition prior to the disposition of the fat-containing composition on the lobster tail.

The Cannabis containing microwaveable lobster tail should be prepared no differently from the non-Cannabis containing lobster tail. Cannabis containing microwaveable lobster tail is recommended to be microwaved for about 3 minutes at full power in a 1100 watt microwave oven. However, adjustments should be made for the size of the lobster tail and the power of the microwave oven used.

The Cannabis containing microwaveable lobster tail is ready for consumption after microwaving. The Cannabis containing microwaveable lobster tail is expected to deliver a psychoactive dose of THC to an adult human after oral consumption. Peak plasma dose of THC after oral consumption generally occurs between 1-2 hours. Two or more plasma peaks is not uncommon. Patients should be advised that oral consumption generally results in higher peak plasma values than consumption by inhalation. Also, peak plasma values are maintained for longer durations after oral consumption than through inhalation.

While not wishing to be bound by theory, it is thought that the THCA of the raw Cannabis disposed in the fat-containing composition of Cannabis containing microwaveable lobster tail is converted to THC nearly completely despite the short cooking duration. The reasons for this are particularly to the Cannabis containing microwaveable lobster tail product.

Microwave cooking is well known to active water-molecule in food. Microwave cooking is also well known for creating hotspots due to the relatively long waves of the microwave. Therefore, effected water molecules within the fat-containing composition are rapidly excited to high temperatures ˜145° C. (293° F.). The energy is dispersed into the THCA and fat. The fat liquefies as it absorbs the heat of the water. The THCA is decarboxylated as it absorbs the heat of the water molecules. However, the lobster meat next to the fat-containing composition acts as a heat sink and ultimately absorbs much of the energy from the excited water molecule. Early in the microwave cooking process, the lipids may return to a solid-state after dispersing the energy.

The process repeats water molecules in the fat-containing composition are excited to an above boiling temperature, the heat is dispersed to the surrounding fat and THCA and ultimately to the adjacent lobster meat. The resulting TCH is readily dissolved and stabilized in the fat-containing composition.

Each cycle converts a portion of the THCA to TCH. After about 3 minutes the fat-containing composition has sustained liquefaction and the lobster meat has reached a temperature of about 60° C. (140° F.). Decarboxylation will continue at this point if any THCA remains in the fat-containing composition, albeit the rate of decarboxylation is reduced from that of active microwaving. During standing-time, after microwaving, residual THCA is further converted to THC.

Alternatively, decarboxylated Cannabis can be added to the microwavable lobster tail product as cannabutter or as processed decarboxylated Cannabis mixed with the fat-soluble substance during the packaging of the microwavable lobster tail product. In this version of the product, the active THC is protected during the heating step by being dissolved in the lipid substance, as well as through proximity to the heat sink of the lobster tail meat.

Regional Specification of Cannabis

It is known that Cannabis has many varieties. Many of these varieties have specific flavors and characteristics that are a product of the region, climate and soil in which the plants are grown as much as the genetics of the seeds.

Growing conditions can also impart characteristics in Cannabis buds. For instance, Cannabis grown hydroponically imparts characteristics on the resulting buds that are distinct from the same seed variety grown in soil. For instance, the now general term for Cannabis, “Chronic”, was coined initially to indicate a hydroponically grown variety.

While the term “terrior” is specific to grapes and wine and refers to all of the factors that go into producing wine grapes in a vineyard, from the climate to the soil to the elevation. Cannabis varieties have a group of factors that go into producing Cannabis buds.

Varieties of Cannabis can therefore be differentiated by region, flavor profile, farmer and strain. The lobsters of this disclosure can be paired with specific Cannabis, herbs and kinds of butter to impart a specific overall dining experience to the consumer.

Cannabutter

Cannabutter is a mixture of butter and decarboxylated cannabidiols, particularly THC and CBD. Cannabutter is commonly used in recipes to provide an oral dosage form of THC and/or CDB.

Care must be taken during the cooking steps as the active TCH can easily be destroyed when heating. During smoking typically a third of the active TCH is lost to combustion. Likewise, TCH is sensitive to heat and can be destroyed during cooking.

Cannabutter is prepared in a variety of methods. Commonly, cannabutter is prepared by taking advantage of the lipophilic property of THC. Butter is melted in boiling water and the temperature is reduced while maintaining the liquefaction of the butter. Previously decarboxylated Cannabis is added to the high-temperature non-boiling water. Over time the highly lipophilic THC is dissolved in the lipid portion of the mixture. After THC extraction, the mixture is then strained to remove the plant material. The mixture is then cooled and the butter is allowed to solidify. The butter is then skimmed from the top of the water. The THC from the Cannabis is now transferred to the butter and can be used in food preparation.

Activated THC and CBD Additive.

THC and CDB can be added directly to the fat-containing composition during the packaging of the microwavable lobster tail product. THC and CBD can be decarboxylated prior to the addition to the fat-containing composition; if residual THCA and/or CBDA are present it is anticipated that any THCA and/or CBDA will be decarboxylated to activate THC and/or CBDA, respectively, during the microwave step of the cooking process. During the microwave step of the cooking process, the active THC and/or CBD are protected during the heating step by being dissolved in the lipid substance, as well as, through proximity to the heat sink of the lobster tail meat.

Dosing

While every user and, perhaps, their physician should consider and determine their own dosage the following dosages are recommended.

Untreated Cannabis is typically used at about a third of a gram per person per use. However, the range is typically between about 0.1 g to about 1 g per person per use. How much Cannabis is determine based on the potency of the Cannabis, the weight of the user, the tolerance of the user and the goal of the use.

TCHA powder is generally used at about around 0.1-1 mg/kg/use. However it is understood that for some user and some treatment goals the range can be as wide as about 0.01-10 mg/kg/use.

Most of the user opt for a dose of CBD oil of anywhere between 20 and 1,500 mg. per use. However, some users may opt for as little as about 2 mg or as much as about 5 g.

Microwaving Guideline from the CFIA

When microwave cooking the Canadian Food Inspection Agency recommends the following safety steps:

Cover food with a microwave-safe lid or plastic wrap that does not touch the food. Covering food in the microwave will help cook it evenly and thoroughly. Leave a small gap so that steam can escape.

Arrange food items in a single layer on microwave-safe cookware for uniform cooking.

Follow your recipe or instructions on the food's packaging. Adjust cooking times based on the power of your microwave. Food will take longer to reach a safe internal temperature in a lower-powered microwave.

Rotate and stir food several times during cooking to ensure that the heat is distributed evenly.

Observe standing times for microwaved food after cooking. Standing times complete the cooking and allow for better heat distribution within the food. Always check the internal temperature before eating.

Safe Ship Conditions During Catch

There is extensive evidence of illness in humans associated with consuming shellfish contaminated by fecal wastes of warm-blooded animals, naturally-occurring bacteria and algal biotoxins. The more common of these illnesses include typhoid, salmonellosis, gastroenteritis, infectious hepatitis, norovirus, Vibrio parahaemolyticus and Vibrio vulnificus infections, paralytic shellfish poisoning, and amnesic shellfish poisoning.

As such operators of shellfish harvesting, handling and/or aquaculture maintenance vessels should consider the following:

Vessels catching lobster for use with the inventions disclosed are expected to have a designated human waste receptacle or holding tank on-board.

Furthermore, Human waste receptacles or holding tanks are to be emptied into approved sewage systems in a manner that will not cause contamination of the shellfish or shellfish harvest area.

Vessels catching lobster for use with the inventions disclosed are expected to comply with any other relevant federal, provincial, or territorial legislation or requirements.

Lobsters are to be rinsed reasonably free of sediments and detritus as soon after harvesting as is feasible, using water from approved shellfish harvest areas. Lobsters may be rinsed at the time of harvest at the harvest area.

The temperature of lobster is to be controlled during transport when ambient air temperature and time of travel are such that unacceptable bacterial growth or deterioration may occur. Specific handling requirements must be followed by licensed operators and is dictated by licensing authorities to control undesirable growth of pathogens such as Vibrio species.

Cannabidiol

Cannabidiol (commonly referred to as “CBD” and “CDB oil” all terms are used interchangeably herein) is a major phytocannabinoid present in the Cannabis sativa plant. It lacks the psychotomimetic and other psychotropic effects that the main plant compound tetrahydrocannabinol (THC). Cannabidiol while lacking these effects is also shown to antagonize THC. It is now well established, that CBD has therapeutic potential over a wide range of non-psychiatric and psychiatric disorders such as anxiety, depression and psychosis.

In vivo studies indicate that these benefits while unitary depend on the behavioral response of the patient and the effect measured. While not wishing to be bound by theory, acute anxiolytic and antidepressant-like effects seem to rely mainly on facilitation of 5-HT1A-mediated neurotransmission in key brain areas related to defensive responses, including the dorsal periaqueductal grey, bed nucleus of the stria terminalis and medial prefrontal cortex. Other effects, such as anti-compulsive, increased extinction and impaired reconsolidation of aversive memories, and facilitation of adult hippocampal neurogenesis could depend on potentiation of anandamide-mediated neurotransmission. Finally, activation of TRPV1 channels may help to explain the antipsychotic effect and the bell-shaped dose-response curves commonly observed with cannabidiol. Various studies indicate other possible neuroactive mechanisms including inhibition of adenosine uptake, inverse agonism at CB2 receptor, CB1 receptor antagonism, GPR55 antagonism, PPARg receptors agonism, intracellular (Ca2 [symbol] ) increase, etc.). Given, its safety profile and a wide range of therapeutic potential, cannabidiol has great potential to alieve many human anxieties.

Cannabidiol is the main non-psychotropic phytocannabinoid present in the Cannabis sativa plant, constituting up to 40 percent of its extract. CBD is a safe compound with a wide range of therapeutic applications, including the treatment of psychiatric disorders. These findings make this substance an attractive candidate for therapeutic use. However, such use has some limiting factors. In addition to its low and variable oral bioavailability in humans, pure cannabidiol possesses a narrow therapeutic dose range. It would be desirable to have a wider therapeutic dose range and a more bioavailable form of cannabidiol. Such a formulation is presently presented being topical formulations containing cannabidiol and a cultivated blend of carriers and adjuvants to transdermally deliver active cannabidiol in a therapeutic dose.

Film

Most microwavable films can be modified for use with the invention. Preferably, films are temperature-safe to 205° C. (400° F.). Preferred bags are made of highly permeable films, which are produced in a co-extrusion process have a guaranteed oxygen transmission rate (OTR) of no less than 10,000 cc/m²/24 hr at STP. This permeability allows aerobic spoilage organisms to grow and spoil the product before any toxin is produced under moderate abuse temperatures. These preferred films are ASTM D3985 compliant to the international standard. Furthermore, the film of the preferred packaged microwaveable lobster tails complies with the rules and regulations of the Canadian Food Inspection Agency. Still, further, the films of the preferred bags preferably comply with the US Food and Drug Administration guidance as published in Fish and Fisheries Products Hazard and Control Guidance. Also, preferably the films meet or exceed the current standards of the World Health Organization.

Labeling

It is preferred that packaged microwaveable lobster tails are labeled according to the rules and regulations of the Canadian Food Inspection Agency. More preferably, packaged microwaveable lobster tails are labeled “Frozen Lobster Meat” and/or “chair de homard congelée”. It is also preferred that the packaged microwaveable lobster tails comply with the packaging requirement of the US Food and Drug Administration. Also preferably the labels comply with the current standards of the World Health Organization.

Values

Unless explicitly stated otherwise, each numerical value and range should be interpreted as being approximate as if the word “about” or “approximately” preceded the value or range.

Scope of Disclosure

It should be understood that various changes in the details, materials, and arrangements of the parts which have been described and illustrated in order to explain the nature of this invention may be made by those skilled in the art without departing from the scope of the invention. For example, it should be understood that the inventive concepts of embodiments of the invention may be applied to foods other than lobster.

Although the disclosure is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the invention.

Although the elements in the following method claims, if any, are recited in a particular sequence with corresponding labeling unless the claim recitations otherwise imply a particular sequence for implementing some or all of those elements, those elements are not necessarily intended to be limited to being implemented in that particular sequence.

Although the invention has been set forth in terms of the exemplary embodiments described herein and illustrated in the attached documents, it is to be understood that such invention is purely illustrative and is not to be interpreted as limiting. Consequently, various alterations, modifications, and/or alternative embodiments and applications may be suggested to those skilled in the art after having read this disclosure. Accordingly, it is intended that the invention be interpreted as encompassing all alterations, modifications, or alternative embodiments and applications as fall within the true spirit and scope of this disclosure.

It will be further understood that various changes in the details, materials, and arrangements of the parts which have been described and illustrated in order to explain the nature of this disclosure may be made by those skilled in the art without departing from the scope of the disclosure as expressed in the following claims.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

### EXAMPLES

### Example 1: Lobster Tail with Cannabis

The following embodiment is a microwaveable lobster tail containing raw Cannabis.

Starting with eight lobsters freshly caught in Maine waters. In cool conditions, near 0 C (32 F), the tails were removed the lobster. Each tail was about 120 g (4 oz) give or take about 7 grams (¼ oz). The shell was kept intact.

In a separate room and normal, slightly warm, conditions (25 C, 77 F), a heavy cup of butter (227 g) was softened. Cannabis was selected for a high concentration of TCHA amounting to about 8 grams. The Cannabis was recently harvested, dried and otherwise untreated. The Cannabis was kneaded into the softened butter and scooped in to eight equal portions (about 30 g each).

Returning to the lobster tails a cut was made split the shell more than half lengthwise on the top of the shell for each tail. After splitting the tail meat was lifted in the shell to create a surface for the butter to sit and to ensure sufficient contact between the butter/Cannabis and the lobster tail meat

The prepared butter and Cannabis combination was deposited on the exposed portion of lobster meat.

The lobster tails were flash frozen with a chilled gas well below −10 C. The gas was applied until the tails were about −7 C (−20 F).

Each frozen lobster tails including butter was placed in a pouch made of microwavable film. The pouches were sealed under vacuum. The lobster tails were stored for at least 24 hours at −7 C (−20 F).

Without thawing, one of the microwaveable pouches was pierced the package at least once; a single pouch was placed in a typical residential microwave. The microwave was turned on full power for about three minutes with a rotating plate. The butter was observed to liquefy.

After cooling for a minute, the lobster tail was removed from the pouch and placed in a bowl. The remaining butter, including Cannabis, was poured into the bowl over the tail.

The lobster tail and the butter were then plated on standard plate and seasoned with butter. It was served with a French Baguette to allow all the butter to be absorbed and consumed

The lobster tail and butter were completely consumed by a guest. The guest reported a delicious lobster taste and experiencing a euphoric high equal or exceeding that associated with consuming a gram of marijuana by water-pipe.

### Example 2: Lobster Tail and THCA

Under normal, slightly warm, conditions (25 C, 77 F), a heavy cup of butter (227 g) was softened. THCa powder was obtained from a dispensary, 60 mg. The powder was stirred into the softened butter. After being fully mixed together, the butter was scooped i to eight equal portions (about 30 g each containing about 12 mg of powdered THCa).

Lobster tails were prepared as in Example 1 with the substitution of THCa containing butter for the Cannabis-containing butter.

The guest who consumed the lobster tail described a delicious lobster flavor. The powder was imperceptible by taste, but the effect was a noticeable strong high equal to smoked Cannabis.

### Example 3: Lobster Tail and CBD Oil

Under normal, slightly warm, conditions (25 C, 77 F), a heavy cup of butter (227 g) was softened. CBD oil was obtained from a dispensary. 2.5 mL was stirred into the softened butter. After being fully mixed together, the butter was scooped into eight equal portions (about 30 g each containing about 300 mg CDB oil).

Lobster tails were prepared as in Example 1 with the substitution of CBD oil containing butter for the Cannabis-containing butter.

The guest who consumed the lobster tail described a delicious lobster flavor. The CBD oil was imperceptible by taste, but the effect was a noticeable calm typical of buccal administered CBD oil.

1. A microwaveable lobster product comprising:
A substantially uncooked lobster tail having a shell containing lobster meat, the shell having a longitudinal axis with a dorsal side and a ventral side with at least two cuts formed generally parallel to the longitudinal axis;
Wherein at least one cut is on the dorsal side of the lobster tail; and,
Wherein at least one cut is on the ventral side of the lobster tail;
Wherein the lobster meat is at least partially split generally parallel to the longitudinal axis;
A fat-containing composition disposed in a region above the lobster meat and below the lobster tail shell; and
A microwaveable package comprising first and second resilient layers, the first resilient layer disposed under the lobster tail and contacting the lobster tail, and the second resilient layer disposed above the lobster tail and contacting the lobster tail and the fat-containing composition; wherein the first and second resilient layer are non-rigid;
Wherein the microwaveable lobster product is frozen to a temperature of at least −18° C.

2. The microwaveable lobster product of claim 1, wherein the fat-containing composition is disposed above an exposed portion of lobster meat on the ventral side of the lobster tail.

3. The microwaveable lobster product of claim 1, wherein the fat-containing composition is disposed above at least one cut formed along the longitudinal axis of the shell on the ventral side of the lobster tail.

4. The microwaveable lobster product of claim 1, wherein the microwaveable package is vacuum-packed.

5. The microwaveable lobster product of claim 1, wherein the fat-containing composition is a solid or semi-solid compound.

6. The microwaveable lobster product of claim 1, further comprising a second lobster tail having a shell containing lobster meat, the shell having a longitudinal axis with at least two cuts formed generally parallel to the longitudinal axis;
Wherein the lobster meat is at least partially split generally parallel to the longitudinal axis;
Wherein:
the first resilient layer is disposed under both lobster tails and contacts both lobster tails,
the second resilient layer is disposed above both lobster tails and contacts both lobster tails and the fat-containing composition, and
the solid or semi-solid fat-containing composition is disposed in a region above both lobster tails.

7. The microwaveable lobster product of claim 1, wherein the lobster tail is unglazed.

8. The microwaveable lobster product of claim 1, wherein the lobster tail is unbrined.

9. The microwaveable lobster product of claim 1, wherein the fat-containing composition is composed primarily of unsalted butter.

10. A method for manufacturing a microwaveable lobster product, the method comprising:
(a) Obtaining a substantially uncooked lobster tail having a shell containing lobster meat, the shell having a longitudinal axis with a dorsal side and a ventral side
(b) forming at least two cuts in the shell of the lobster tail each generally parallel to the longitudinal axis of the shell of a lobster tail containing lobster meat;
Wherein at least one cut is on the dorsal side of the lobster tail; and,
Wherein at least one cut is on the ventral side of the lobster tail;
(b) forming at least one cut in the lobster tail meat generally parallel to the longitudinal axis wherein the lobster tail meat is at least partially split;
(c) disposing a fat-containing composition in a region above the lobster tail on the ventral side of the lobster tail; and
(d) disposing the lobster tail and fat-containing composition together in a microwaveable package comprising first and second resilient layers, such that the first resilient layer is disposed under the lobster tail and contacts the lobster tail, and the second resilient layer is disposed above the lobster tail and contacts the lobster tail and the fat-containing composition; wherein the first and second resilient layer are non-rigid;
(e) freezing the microwaveable package containing the lobster tail to at least 18° C.

11. The method of claim 10, wherein step (c) comprises disposing the fat-containing composition above an exposed portion of lobster meat.

12. The method of claim 10, wherein step (c) comprises disposing the fat-containing composition above at least one cut formed along the longitudinal axis of the shell.

13. The method of claim 10, further comprising (f) vacuum-packing the microwaveable package.

14. The method of claim 10, wherein the fat-containing composition is a solid or semi-solid compound.

15. The method of claim 10, further comprising:
(f) disposing, in the microwaveable package, a second lobster tail having a shell containing lobster meat, the shell having a longitudinal axis with at least two cuts formed along the longitudinal axis:
Wherein at least one cut is on the dorsal side of the lobster tail; and,
Wherein at least one cut is on the ventral side of the lobster tail;
Wherein the lobster tail meat has at least one cut generally parallel to the longitudinal axis wherein the lobster tail meat is at least partially split;
Wherein,
the first resilient layer is disposed under both lobster tails and contacts both lobster tails,
the second resilient layer is disposed above both lobster tails and contacts both lobster tails and the fat-containing composition, and
the solid or semi-solid fat-containing composition is disposed in a region above both lobster tails.

16. The method of claim 10, wherein the lobster tail is unglazed.

17. The method of claim 10, wherein the lobster tail is unbrined.

18. The microwaveable lobster product of claim 10, wherein the fat-containing composition is composed primarily of unsalted butter.

19. The method of claim 13, comprising the step of (g) delivering the lobster tail from the site of freezing to site of a distributor while maintaining a temperature of at least −18° C.

20. The method of claim 10, wherein the lobster tail is frozen to at least −25° C.

21. The method of claim 10, wherein the lobster tail is frozen to at least −75° C.

22. The method of claim 10, wherein the substantially uncooked lobster was obtained from a catch of lobsters caught on a vessel having a designated human waste holding tank on-board

23. A method of cooking a lobster tail comprising the following steps:
(a) obtaining microwaveable lobster product comprising:
(1) A substantially uncooked lobster tail having a shell containing lobster meat, the shell having a longitudinal axis with a dorsal side and a ventral side with at least two cuts formed generally parallel to the longitudinal axis;
Wherein at least one cut is on the dorsal side of the lobster tail; and,
Wherein at least one cut is on the ventral side of the lobster tail;
Wherein the lobster meat is at least partially split generally parallel to the longitudinal axis;
(2) a fat-containing composition disposed in a region above the lobster tail; on the ventral side of the tail and
(3) a microwaveable package comprising first and second resilient layers, the first resilient layer disposed under the lobster tail and contacting the lobster tail, and the second resilient layer disposed above the lobster tail and contacting the lobster tail and the fat-containing composition; wherein the first and second resilient layer are non-rigid;
(b) Piercing the microwaveable package at least once;
(c) Inserting the microwaveable package containing the lobster tail into the interior of a microwave device;
(d) Operating the microwave device so that the interior of the lobster reaches a temperature such that the fat-containing composition liquefies; and
(e) Removing the package containing the lobster tail from the microwave device.

24. The method of claim 23, wherein during step (d) the lobster meat in the interior of the lobster tail reaches a temperature of at least about 60° C.

25. A microwaveable lobster product comprising:
a lobster tail having a shell containing lobster meat, the shell having a longitudinal axis with at least one cut formed generally parallel to the longitudinal axis;
a fat-containing composition disposed in a region above the lobster tail;
Wherein the fat-containing is mixed with at least one Cannabis cannabinoid and
a microwaveable package comprising first and second resilient layers, the first resilient layer disposed under the lobster tail and contacting the lobster tail, and the second resilient layer disposed above the lobster tail and contacting the lobster tail and the fat-containing composition.

26. The microwaveable lobster product of claim 25, wherein the at least one Cannabis cannabinoid is THC.

27. The microwaveable lobster product of claim 25, wherein the at least one Cannabis cannabinoid is THCA.

28. The microwaveable lobster product of claim 25, wherein the at least one Cannabis cannabinoid is CBD.

29. The microwaveable lobster product of claim 25, wherein the at least one Cannabis cannabinoid is CBDA

30. The microwaveable lobster product of claim 25, wherein the at least one Cannabis cannabinoid is contained in untreated Cannabis plant material.

31. The microwaveable lobster product of claim 25, wherein the at least one Cannabis cannabinoid is contained Cannabis plant material that has undergone decarboxylation.

32. The microwaveable lobster product of claim 1, wherein the product is labeled, “frozen lobster meat”.

33. The microwaveable lobster product of claim 1, wherein the product is labeled, “chair de homard congelée”.

34. The microwaveable lobster product of claim 1, wherein at least one resilient layer has an oxygen transmission rate of no less than 10,000 cc/m²/24 hr at STP.

35. The microwaveable lobster product of claim 1, wherein both resilient layers have an oxygen transmission rate of no less than 10,000 cc/m²/24 hr at STP.
